(19) United States
(12) Patent Application Publication
(10) Pub. No.: US 2024/0115581 A1
(43) Pub. Date: Apr. 11, 2024

(54) Combination therapy for the treatment or prevention of neurological disorders

(71) Applicant: Norwegian University of Science and Technology (NTNU), Trondheim (NO)

(72) Inventors: Christiana Bjorkli, Trondheim (NO); Mary Hemler, Trondheim (NO); Joshua Julian, Trondheim (NO); Axel Sandvig, Trondheim (NO); Ioanna Sandvig, Trondheim (NO)

(21) Appl. No.: 17/951,747

(22) Filed: Sep. 23, 2022

(51) Int. Cl. A61K 31/551; A61K 31/4545; A61K 49/00; A61P 25/28

(52) U.S. Cl. CPC A61K 31/551; A61K 31/4545; A61K 49/0008; A61P 25/28

(57) ABSTRACT

The present invention relates to products and methods for the treatment and/or prevention of neurological disorders characterized by neurodegeneration. More particularly, the invention provides a product comprising: (i) a Rho-associated protein kinase (ROCK) inhibitor (e.g. fasudil) or a pharmaceutically acceptable salt thereof; and (ii) a farnesyltransferase inhibitor (e.g. lonafarnib) or a pharmaceutically acceptable salt thereof; for use in treating or preventing a neurological disorder characterized by neurodegeneration in a subject.

### FIELD OF THE INVENTION

The present invention relates to products and methods for the treatment and/or prevention of neurological disorders characterized by neurodegeneration. More specifically, the present invention relates to combination therapies comprising Rho-associated protein kinase (ROCK) inhibitor (e.g. fasudil) compositions in combination with farnesyltransferase inhibitor (e.g. lonafarnib) compositions for use in the treatment and/or prevention of neurological disorders characterized by neurodegeneration (e.g. neurodegenerative diseases), such as Alzheimer's disease. Methods for treating and/or preventing of neurological disorders characterized by neurodegeneration comprising administering ROCK inhibitor (e.g. fasudil) compositions in combination with farnesyltransferase inhibitor (e.g. lonafarnib) compositions are also provided. Pharmaceutical compositions and kits comprising the aforementioned compositions and their use in the treatment and/or prevention of neurological disorders characterized by neurodegeneration are also disclosed.

### BACKGROUND OF THE INVENTION

Neurodegenerative disorders encompass a wide range of conditions that result from progressive damage to neurons and neuronal connections that are essential for mobility, coordination, strength, sensation, and cognition. Every three seconds someone is diagnosed with dementia, and Alzheimer's disease (AD) constitutes most of these cases.

Researchers have made tremendous strides in research to understand the origin of AD, and we now know that the neurons in lateral entorhinal cortex (LEC) layer II (a small brain region in the temporal lobes) are at selective risk for neurodegeneration in patients going through transition stages to AD (referred to as mild cognitive impairment [MCI]), and during this transition stage as much as half of the cell population is lost. The AD progression in humans has been well-characterized by biomarker studies to assess pathological hallmarks at various stages of the disease.

Biomarkers in AD can be elucidated by cerebrospinal fluid (CSF) analysis (commonly used biomarkers include decreased amyloid-β (Aβ)₄₂, increased total tau, and increased phosphorylated tau) or neuroimaging markers of disease, such as positron emission tomography (PET) revealing amyloid plaques and tau pathology.

AD leads to a gradual and eventual irreversible loss of neurons and synapses. Progressive cortical atrophy is the main gross anatomical correlate of AD and is most prominent in the frontal, parietal, and temporal lobes, with relative sparing of occipital, and primary motor and sensory regions. Atrophy of the hippocampus is prominent and can extend to the amygdala. The ventricles, particularly the temporal horns, are frequently enlarged. Notably however, none of these features are specific to AD.

The most remarkable features of AD are the stereotypic patterns by which amyloid plaques and neurofibrillary tangles (NFTs) appear throughout the brain, and that toxic, misfolded Aβ and tau can serve as templates that convert their innocuous counterparts into equivalent pathological forms both in vitro and in vivo. Pathological tau is also involved in other diseases like progressive supranuclear palsy, corticobasal degeneration, Pick's disease and frontotemporal degeneration (FTLD). Pathological Aβ is also seen in cerebral amyloid angiopathy, vascular dementia and Down's syndrome (DS).

AD is unique in the fact that it is characterized by the misfolding of otherwise unrelated proteins, Aβ and tau, causing distinct histopathological changes that converge into the amyloid plaque, which is composed of Aβ deposits, surrounded by degenerating neurites accumulating tau protein.

The basis of the amyloid cascade hypothesis, which forms the backbone of the current understanding of the pathogenesis of AD, is that accumulation of Aβ is an early event leading to neurodegeneration. Aβ peptides are composed of various amino acids and generated through proteolytic cleavage of APP by several enzyme complexes, secretases.

Cleavage by α-secretase and subsequently by the γ-secretase complex forms nonamyloidogenic products of APP. An alternative amyloidogenic pathway, with cleavage of APP first by the β-secretase and subsequently by the γ-secretase complex, leads to an accumulation of insoluble Aβ proteins in the brain. APP cleavage through β-secretase and γ-secretase can produce several isoforms of Aβ, of which the 40 and 42 amino acid forms are the most prominent. Aβ₄₀ is considerably less prone to oligomerization (i.e., the process of aggregating into oligomers from which larger, insoluble fibrils are formed) compared to Aβ₄₂, and is regarded as less neurotoxic.

Intraneuronal Aβ accumulation has been identified in AD patients, transgenic mice, and cultured cells, has been found to appear prior to extracellular amyloid plaque formation and results in synaptic dysfunction.

In patients with mild cognitive impairment (MCI), intraneuronal Aβ immunoreactivity has been reported in brain regions that are more prone to the development of early AD pathology, such as the hippocampus and the entorhinal cortex (EC). Because the accumulation of intraneuronal Aβ has been shown to precede extracellular amyloid plaque formation, and intraneuronal Aβ levels decrease once amyloid plaques accumulate, it has been suggested that the build-up of intraneuronal Aβ is an early event in the progression of AD.

Amyloid plaques are largely composed of the Aβ peptide. Neuritic (so-called dense-core) plaques have a dense centre of amyloid surrounded by a halo of silver-positive neurites. Dense-core plaques frequently include neuronal and glial cellular elements. After the sequencing of the peptide (to determine the amino acids that make up Aβ) and development of antibodies, it was found that Aβ also aggregates in ‘diffuse’ plaques of several different morphologies. Diffuse plaques are much less dense and consist of non-fibrillary forms of Aβ, are only visible with immunohistochemical techniques, and are hypothesized to represent an early stage in the formation of amyloid plaques.

Crucially, Aβ oligomers instigate tau hyperphosphorylation, which eventually leads to the formation of NFTs. Co-occurring with extracellular Aβ accumulation, the intracellular build-up of twisted filaments (pre-tangles), leads to accumulations of abnormal tau in dendrites (i.e., neuropil threads) and cell somata of selected neuronal populations (NFTs).

Tau is a microtubule-binding protein found largely in axons where it serves to stabilize microtubules. During the course of AD, tau is hyperphosphorylated, becomes detached from microtubules, and accumulates in the somatodendritic compartment as paired helical filaments and straight filaments. The deposition of NFTs occur in a hierarchical fashion beginning in the superficial lateral EC (LEC) and progressing through the hippocampus, association cortices, and only affecting primary sensory areas in late stages of the disease. The brain density of NFTs directly correlates with the degree of dementia in patients.

Despite comprehensive research and a large number of therapeutic trials, there is still no curative treatment for AD. Disease modifying treatments available today are designed to delay symptoms and cognitive decline in symptomatic patients. However, the blood-brain barrier (BBB) remains a major challenge for engaging pharmaceutical drug targets in the brain. Accordingly, there is a need for new treatments for AD and numerous other neurological disorders associated with neurodegeneration. However, before any new treatments can reach the clinic, they first need to be tested in preclinical models using basic research strategies. In this respect, there is also a desire for robust preclinical models with improved comparability with the human condition, and thereby improved translatability into the clinic.

### SUMMARY OF THE INVENTION

In work leading to the present disclosure, the inventors have developed and characterised a transgenic mouse model that finds utility in the investigation neurological disorders associated with neurodegeneration, particularly disorders characterised by the accumulation of Aβ and aggregation of tau within the brain, particularly the synergistic effects of these proteins in AD. This led to the development of a preclinical system and method for assessing and/or evaluating the effects of drug candidates in a neurological disease model. Validation of the system and model involved the evaluation of drug candidates that had previously been approved for use in treating other diseases, fasudil and lonafarnib. Unexpectedly, the inventors determined that these repurposed drugs were particularly effective at reducing biomarkers associated with neurodegenerative diseases, such as t-tau, Aβ₄₀ and Aβ₄₂ when used in combination. Moreover, these biomarker findings translated to an improvement of cognitive defects usually associated with neurodegenerative diseases, such as AD.

Accordingly, provided herein is a product comprising:
(i) a Rho-associated protein kinase (ROCK) inhibitor or a pharmaceutically acceptable salt thereof; and
(ii) a farnesyltransferase inhibitor or a pharmaceutically acceptable salt thereof;
for use in treating or preventing a neurological disorder characterized by neurodegeneration in a subject.

Further provided herein is a product comprising:
(i) fasudil or a pharmaceutically acceptable salt thereof; and
(ii) lonafarnib or a pharmaceutically acceptable salt thereof;
for use in treating or preventing a neurological disorder characterized by neurodegeneration in a subject.

Alternatively viewed, provided herein is a product comprising:
(i) a Rho-associated protein kinase (ROCK) inhibitor or a pharmaceutically acceptable salt thereof; and
(ii) a farnesyltransferase inhibitor or a pharmaceutically acceptable salt thereof;
for use in inhibiting or arresting (i.e. slowing, reducing or attenuating) the progression of cognitive impairment in a subject, e.g. a subject having or showing signs of neurodegeneration, e.g. a subject with mild cognitive impairment.

Further provided herein is a product comprising:
(i) fasudil or a pharmaceutically acceptable salt thereof; and
(ii) lonafarnib or a pharmaceutically acceptable salt thereof;
for use in inhibiting or arresting (i.e. slowing, reducing or attenutating) the progression of cognitive impairment in a subject, e.g. a subject having or showing signs of neurodegeneration, e.g. a subject with mild cognitive impairment.

Another aspect provided herein is a method of treating or preventing a neurological disorder characterized by neurodegeneration in a subject, the method comprising administering to the subject a therapeutically effective amount of: (i) a Rho-associated protein kinase (ROCK) inhibitor or a pharmaceutically acceptable salt thereof; and (ii) a farnesyltransferase inhibitor or a pharmaceutically acceptable salt thereof.

Also provided herein is a method of inhibiting or arresting (i.e. slowing, reducing or attenuating) the progression of cognitive impairment in a subject (e.g. a subject having or showing signs of neurodegeneration, e.g. a subject with mild cognitive impairment), the method comprising administering to the subject a therapeutically effective amount of: (i) a Rho-associated protein kinase (ROCK) inhibitor or a pharmaceutically acceptable salt thereof; and (ii) a farnesyltransferase inhibitor or a pharmaceutically acceptable salt thereof.

Further provided herein is a method of treating or preventing a neurological disorder characterized by neurodegeneration in a subject, the method comprising administering to the subject a therapeutically effective amount of: (i) fasudil or a pharmaceutically acceptable salt thereof; and (ii) lonafarnib or a pharmaceutically acceptable salt thereof.

Also provided herein is a method of inhibiting or arresting (i.e. slowing, reducing or attenuating) the progression of cognitive impairment in a subject (e.g. a subject having or showing signs of neurodegeneration, e.g. a subject with mild cognitive impairment), the method comprising administering to the subject a therapeutically effective amount of: (i) fasudil or a pharmaceutically acceptable salt thereof; and (ii) lonafarnib or a pharmaceutically acceptable salt thereof.

Yet further provided herein is the use of: (i) a Rho-associated protein kinase (ROCK) inhibitor or a pharmaceutically acceptable salt thereof; and (ii) a farnesyltransferase inhibitor or a pharmaceutically acceptable salt thereof; in the manufacture of a product for treating or preventing a neurological disorder characterized by neurodegeneration in a subject.

Still further provided herein is the use of: (i) fasudil or a pharmaceutically acceptable salt thereof; and (ii) lonafarnib or a pharmaceutically acceptable salt thereof; in the manufacture of a product for treating or preventing a neurological disorder characterized by neurodegeneration in a subject.

Also provided herein is the use of: (i) a Rho-associated protein kinase (ROCK) inhibitor or a pharmaceutically acceptable salt thereof; and (ii) a farnesyltransferase inhibitor or a pharmaceutically acceptable salt thereof; in the manufacture of a product for inhibiting or arresting (i.e. slowing, reducing or attenuating) the progression of cognitive impairment in a subject (e.g. a subject having or showing signs of neurodegeneration, e.g. a subject with mild cognitive impairment).

Still further provided herein is the use of: (i) fasudil or a pharmaceutically acceptable salt thereof; and (ii) lonafarnib or a pharmaceutically acceptable salt thereof; in the manufacture of a product for inhibiting or arresting (i.e. slowing, reducing or attenuating) the progression of cognitive impairment in a subject (e.g. a subject having or showing signs of neurodegeneration, e.g. a subject with mild cognitive impairment).

Further provided is a kit comprising:
(i) a Rho-associated protein kinase (ROCK) inhibitor or a pharmaceutically acceptable salt thereof (e.g. fasudil or a pharmaceutically acceptable salt thereof); and
(ii) a farnesyltransferase inhibitor or a pharmaceutically acceptable salt thereof (e.g. lonafarnib or a pharmaceutically acceptable salt thereof), optionally wherein (i) and (ii) are for separate, simultaneous or sequential use: (a) to treat or prevent a neurological disorder characterized by neurodegeneration; or (b) to inhibit or arrest (i.e. slow, reduce or attenuate) the progression of cognitive impairment in a subject (e.g. a subject having or showing signs of neurodegeneration, e.g. a subject with mild cognitive impairment).

Also provided herein is a pharmaceutical composition comprising:
(i) a Rho-associated protein kinase (ROCK) inhibitor or a pharmaceutically acceptable salt thereof (e.g. fasudil or a pharmaceutically acceptable salt thereof); and
(ii) a farnesyltransferase inhibitor or a pharmaceutically acceptable salt thereof (e.g. lonafarnib or a pharmaceutically acceptable salt thereof), optionally wherein the pharmaceutical composition is for use in: (a) treating or preventing a neurological disorder characterized by neurodegeneration; or (b) inhibiting or arresting (i.e. slowing, reducing or attenuating) the progression of cognitive impairment in a subject (e.g. a subject having or showing signs of neurodegeneration, e.g. a subject with mild cognitive impairment).

Still further provided herein is a system for assessing the effect of one or more candidate drugs in a neurological disorder animal model comprising:
(a) a transgenic animal model having physiological characteristics associated with a neurological disorder characterized by neurodegeneration;
(b) means for administering said one or more candidate drugs intracerebroventricularly to the transgenic animal model; and optionally
(c) means for obtaining a sample from the transgenic animal model for analysis of one or more biomarkers associated with a neurological disorder characterized by neurodegeneration;
wherein the transgenic animal model has been additionally modified to express exogenous tau protein in neurons.

Yet further provided herein is a method for assessing the effect of one or more candidate drugs in a neurological disorder animal model comprising:
(a) providing a transgenic animal model having physiological characteristics associated with a neurological disorder characterized by neurodegeneration;
(b) modifying the transgenic animal model of (a) to express exogenous tau protein in neurons;
(c) administering said one or more candidate drugs intracerebroventricularly to the transgenic animal model; and
(d) analysing one or more biomarkers associated with a neurodegenerative disease in a sample obtained from the transgenic animal and/or assessing the cognitive ability and/or behaviour of the transgenic animal, thereby assessing the effect of the one or more candidate drugs.

### DETAILED DESCRIPTION

Rho-associated protein kinase (ROCK) is a kinase belonging to the AGC (PKA/PKG/PKC) family of serine-threonine specific protein kinases. It is primarily involved in regulating the shape and movement of cells by acting on the cytoskeleton. ROCKs (ROCK1 and ROCK2) occur in mammals (human, rat, mouse, cow), zebrafish, Xenopus, invertebrates (C. elegans, mosquito, Drosophila) and chicken. Human ROCK1 has a molecular mass of 158 kDa and is a major downstream effector of the small GTPase RhoA. Mammalian ROCK consists of a kinase domain, a coiled-coil region and a Pleckstrin homology (PH) domain, which reduces the kinase activity of ROCKs by an autoinhibitory intramolecular fold if RhoA-GTP is not present.

The terms “Rho-associated protein kinase (ROCK) inhibitor” and “Rho-associated protein kinase (ROCK) antagonist” are used interchangeably herein and refer to agents capable of directly or indirectly inhibiting, reducing or blocking the activity or function of ROCK. Such agents may work via competitive inhibition, uncompetitive inhibition, on-competitive inhibition or mixed inhibition. A ROCK inhibitor may disrupt the interaction between ROCK and its substrate(s). Preferably, the ROCK inhibitor directly inhibits, reduces or blocks the activity or function of ROCK. Inhibition of ROCK may function to block the Wnt-planar cell polarity (Wnt-PCP) pathway. Thus, the ROCK inhibitor may function to indirectly inhibit the Wnt-planar cell polarity (Wnt-PCP) pathway.

The ROCK inhibitor may be fasudil or a pharmaceutically acceptable salt thereof. Fasudil (5-(1,4-Diazepane-1-sulfonyl)isoquinoline) is a selective RhoA/Rho Kinase (ROCK) inhibitor and has been approved for the treatment of cerebral vasospasm, commonly due to subarachnoid hemorrhage. Fasudil has the structure indicated below. The term “fasudil” as used herein includes pharmaceutically acceptable salts thereof. The pharmaceutically acceptable salt for use as described herein may be the hydrochloride salt.

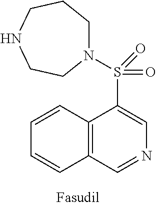 [SMILES: O=S(=O)(c1cncc2ccccc12)N1CCCNCC1]

Pharmaceutical compositions of fasudil are well-known in the art, e.g. WO 2005/117896 and WO2022/086581 (both incorporated herein by reference) and any such compositions may be used in the methods, compositions and uses disclosed herein.

Farnesyltransferase (EC 2.5.1.58) is one of the three enzymes in the prenyltransferase group. Farnesyltransferase (FTase) adds a farnesyl group to proteins bearing a CaaX motif, typically found at the carboxyl terminus of a target protein. Farnesyltransferase's targets include members of the Ras superfamily of small GTP-binding proteins critical to cell cycle progression.

The terms “farnesyltransferase inhibitor” and “farnesyltransferase antagonist” are used interchangeably herein and refer to agents capable of directly or indirectly inhibiting, reducing or blocking the activity or function of farnesyltransferase. Such agents may work via competitive inhibition, uncompetitive inhibition, on-competitive inhibition or mixed inhibition. A farnesyltransferase inhibitor may disrupt the interaction between farnesyltransferase and its protein substrate(s). Preferably, the farnesyltransferase inhibitor directly inhibits, reduces or blocks the activity or function of farnesyltransferase. In particular, the farnesyltransferase inhibitor may function as an autophagic activator or inducer which results in a reduction in the level of misfolded and aggregated proteins. Inhibition of farnesyltransferase may function to block the function or activity of the mTOR pathway. Thus, the farnesyltransferase inhibitor may function to indirectly inhibit the mTOR pathway.

The farnesyltransferase inhibitor may be lonafarnib. Lonafarnib (4-(2-{4-[(11R)-3,10-dibromo-8-chloro-6,11-dihydro-5H-benzo[5,6]cyclohepta[1,2-b]pyridin-11-yl]piperidin-1-yl}-2-oxoethyl)piperidine-1-carboxamide) is a farnesyltransferase inhibitor and has been approved for the treatment of Hutchinson-Gilford progeria syndrome and for the treatment of certain processing-deficient progeroid laminopathies. Typically, lonafarnib is administered orally. Lonafarnib has the structure indicated below. The term “lonafarnib” as used herein includes pharmaceutically acceptable salts thereof. Typically, lonafarnib is provided as a crystalline solid and is may be used in the free drug form.

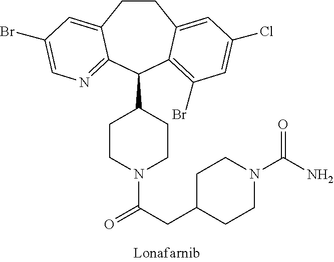 [SMILES: NC(=O)N1CCC(CC(=O)N2CCC([C@H]3c4ncc(Br)cc4CCc4cc(Cl)cc(Br)c43)CC2)CC1]

The terms “agent”, “compound”, and “active” may be used interchangeably herein to refer to a substance that induces a desired pharmacological and/or physiological effect, i.e. inhibition of ROCK or farnesyltransferase, or downstream pathways, i.e. the Wnt-PCP pathway or mTOR pathway. For instance, the farnesyltransferase inhibitor may function to lower the levels of misfolded and aggregated proteins, such as tau. The terms also encompass pharmaceutically acceptable and pharmacologically active forms thereof, including salts.

Pharmaceutically acceptable salts include pharmaceutical acceptable base addition salts and acid addition salts, for example, metal salts, such as alkali and alkaline earth metal salts, ammonium salts, organic amine addition salts, and amino acid addition salts, and sulfonate salts. Acid addition salts include inorganic acid addition salts such as hydrochloride, sulfate and phosphate, and organic acid addition salts such as alkyl sulfonate, arylsulfonate, acetate, benzoate, maleate, fumarate, tartrate, citrate and lactate. Examples of metal salts are alkali metal salts, such as lithium salt, sodium salt and potassium salt, alkaline earth metal salts such as magnesium salt and calcium salt, aluminum salt, and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylat and benzene sulfonic acid salts.

The lists of pharmaceutically acceptable salts listed above apply to all drug substances described herein unless stated otherwise.

The agents described herein may be provided in pharmaceutical composition or formulations comprising a pharmacologically or pharmaceutically acceptable excipient and/or diluent.

“Pharmaceutically acceptable” and “pharmacologically acceptable” as referred to herein refer to ingredients that are compatible with other ingredients used in the methods or uses disclosed herein as well as physiologically acceptable to the recipient. Pharmaceutically acceptable includes that the formulation is sterile and pyrogen free. The carrier, diluent, and/or excipient must be “acceptable” in the sense of being compatible with the active agent and not deleterious to the recipients thereof. Typically, the carriers, diluents, and excipients will be saline or infusion media which will be sterile and pyrogen free, however, other acceptable carriers, diluents, and excipients may be used. In particular, pharmaceutical compositions comprising the agents described herein may be isotonic solutions free of antioxidants, preservatives, and potentially neurotoxic additives. The composition also may be sterile, pyrogen-free and non-autoclavable. Typically, the pH range of the composition may be about 5.0 to about 7.4.

While lonafarnib is sparingly soluble in aqueous buffers, it is preferred that compositions containing lonafarnib do not contain significant levels of dimethyl sulfoxide (DMSO) or dimethyl formamide (DMF), i.e. the compositions may be free or essentially free of DMSO and/or DMF. For example, to prepare a pharmaceutical composition containing lonafarnib, the compound may be dissolved first in ethanol and subsequently diluted with an aqueous buffer, such as phosphate-buffered saline (about pH 7.2). As lonafarnib is not stable in aqueous solutions, it may be necessary to prepare an aqueous solution comprising lonafarnib within 24-48 hours of administration to a subject.

A neurological disorder characterized by neurodegeneration refers to a disease, disorder or injury that involves neuronal damage and/or neuronal cell death. Such disorders typically result in cognitive impairment, such as impaired memory or impaired temporal lobe memory (e.g. memory loss or temporal lobe memory loss). Thus, a neurological disorder characterized by neurodegeneration may refer to a disorder that results in cognitive impairment, particularly progressive cognitive impairment, i.e. cognitive impairment that worsens over time, e.g. due to progressive and irreversible degeneration of neurons. There are many etiologies that underly neurological disorders characterized by neurodegeneration, including oxidative stress and inflammation, which may have numerous preceding causes. Thus, the neurological disease characterized by neurodegeneration may be a neurodegenerative disease, a neurological disorder that affects memory, or a temporal lobe memory disorder.

In particular, a neurological disorder characterized by neurodegeneration may be selected from: dementia, which includes frontotemporal dementia or frontotemporal degeneration, vascular dementia, mixed dementia, dementia with Lewy bodies, semantic dementia and Alzheimer's disease; tauopathy disease; amyotrophic lateral sclerosis (ALS); Parkinson's disease; Spinal muscular atrophy; Pick's disease; Corticobasal syndrome; and normal pressure hydrocephalus. Thus, in a particular aspect the neurological disorder characterized by neurodegeneration may be Alzheimer's disease.

Dementia is a non-specific syndrome (i.e., a set of signs and symptoms) that presents as a serious loss of global cognitive ability in a previously unimpaired person, beyond what might be expected from normal ageing. Dementia may be static as the result of a unique global brain injury. Alternatively, dementia may be progressive, resulting in long-term decline due to damage or disease in the body. While dementia is much more common in the geriatric population, it can also occur before the age of 65. Cognitive areas affected by dementia include, without limitation, memory, attention span, language, and problem solving. Generally, symptoms must be present for at least six months to before an individual is diagnosed with dementia.

Exemplary forms of dementia include frontotemporal dementia (also known as frontotemporal degeneration), Alzheimer's disease, vascular dementia, mixed dementia, semantic dementia, and dementia with Lewy bodies.

Frontotemporal dementia (FTD) is a condition resulting from the progressive deterioration of the frontal lobe of the brain. Over time, the degeneration may advance to the temporal lobe. Second only to Alzheimer's disease (AD) in prevalence, FTD accounts for 20% of pre-senile dementia cases. The clinical features of FTD include memory deficits, behavioral abnormalities, personality changes, and language impairments.

A substantial portion of FTD cases are inherited in an autosomal dominant fashion, but even in one family, symptoms can span a spectrum from FTD with behavioral disturbances, to Primary Progressive Aphasia, to Cortico-Basal Ganglionic Degeneration. FTD, like most neurodegenerative diseases, can be characterized by the pathological presence of specific protein aggregates in the diseased brain (e.g. intraneuronal accumulations of hyperphosphorylated Tau protein in neurofibrillary tangles or Pick bodies).

As noted above, Alzheimer's disease (AD) is the most common form of dementia. There is no cure for the disease, which worsens as it progresses, and eventually leads to death. Most often, AD is diagnosed in people over 65 years of age. However, the less-prevalent early-onset Alzheimer's can occur much earlier.

Common symptoms of Alzheimer's disease include, behavioral symptoms, such as difficulty in remembering recent events; cognitive symptoms, confusion, irritability and aggression, mood swings, trouble with language, and long-term memory loss. As the disease progresses bodily functions are lost, ultimately leading to death. Alzheimer's disease develops for an unknown and variable amount of time before becoming fully apparent, and it can progress undiagnosed for years.

Amyotrophic lateral sclerosis (ALS) (also known as motor neuron disease or Lou Gehrig's disease) refers to a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea).

Parkinson's disease, which may be referred to as idiopathic or primary parkinsonism, hypokinetic rigid syndrome (HRS), or paralysis agitans, is a neurodegenerative brain disorder that affects motor system control. The progressive death of dopamine-producing cells in the brain leads to the major symptoms of Parkinson's. Most often, Parkinson's disease is diagnosed in people over 50 years of age. Parkinson's disease is idiopathic (having no known cause) in most people. However, genetic factors also play a role in the disease.

Symptoms of Parkinson's disease include tremors of the hands, arms, legs, jaw, and face, muscle rigidity in the limbs and trunk, slowness of movement (bradykinesia), postural instability, difficulty walking, neuropsychiatric problems, changes in speech or behavior, depression, anxiety, pain, psychosis, dementia, hallucinations, and sleep problems.

Tauopathy diseases, or Tauopathies, are a class of neurodegenerative disease caused by aggregation of the microtubule-associated protein tau within the brain. Alzheimer's disease (AD) is the most well-known Taupathy disease, and involves an accumulation of tau protein within neurons in the form of insoluble neurofibrillary tangles (NFTs). Other Taupathy diseases and disorders include progressive supranuclear palsy, dementia pugilistica (chromic traumatic encephalopathy), frontotemporal dementia and parkinsonism linked to chromosome 17, Lytico-Bodig disease (Parkinson-dementia complex of Guam), Tangle-predominant dementia, Ganglioglioma and gangliocytoma, Meningioangiomatosis, Subacute sclerosing panencephalitis, lead encephalopathy, tuberous sclerosis, Hallervorden-Spatz disease, lipofuscinosis, Pick's disease, corticobasal degeneration, Argyrophilic grain disease (AGD), Huntington's disease, frontotemporal dementia, and frontotemporal lobar degeneration. Thus, the neurological disorder characterized by neurodegeneration may be selected from any of the aforementioned tauopathies.

As used herein, the term “preventing” includes providing prophylaxis with respect to occurrence or recurrence of a particular disease, disorder, or condition in an individual. An individual may be predisposed to, susceptible to a particular disease, disorder, or condition, or at risk of developing such a disease, disorder, or condition, but has not yet been diagnosed with the disease, disorder, or condition. Thus, preventing a neurological disorder characterized by neurodegeneration may be viewed as inhibiting or arresting (i.e. slowing, reducing or attenuating) the progression of cognitive impairment in a subject, e.g. a subject having or showing signs of neurodegeneration, e.g. a subject with mild cognitive impairment. Thus, the subject may demonstrate a change in their cognition process, i.e. a decline in cognition relative to a comparable healthy (control) subject or to a previous timepoint for the subject, i.e. prior to the onset of neurodegeneration.

Cognitive impairment or deficit as used herein therefore refers to a change (decline or decrease) in the cognition process, i.e. a decline in cognition relative to a comparable healthy subject or to a previous timepoint for the subject, i.e. prior to the onset of neurodegeneration. Assessment of cognition in a subject may be performed using any suitable means known in the art and the determination of a change (e.g. decline) in cognition, i.e. cognitive impairment, may be determined by comparing the assessment with the results of a comparable healthy (control) subject or to the results of an assessment at a previous timepoint for the subject.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

Thus, a subject at risk of developing a neurological disorder characterized by neurodegeneration, as defined above (e.g. Alzheimer's disease), may be a subject with mild cognitive impairment, i.e. a decline in cognition may be a risk factor. Mild cognitive impairment (MCI) refers to an early stage of memory loss or other cognitive ability loss (such as language or visual/spatial perception) in individuals who maintain the ability to independently perform most activities of daily living. MCI may be classified as Amnestic MCI (AMCI) or Nonamnestic MCI (NMCI) and may be assessed or diagnosed using any suitable means known in the art.

AMCI primarily affects memory such that a person may start to forget important information that he or she previously would have recalled easily, such as appointments, conversations or recent events.

NMCI affects thinking skills other than memory, including the ability to make sound decisions, judge the time or sequence of steps needed to complete a complex task, or visual perception.

Thus, a subject at risk of developing a neurological disorder characterized by neurodegeneration may have AMCI and/or NMCI.

Additionally or alternatively, a subject at risk of developing a neurological disorder characterized by neurodegeneration as defined above (e.g. Alzheimer's disease), may be a subject with one or more physiological characteristics (e.g. brain metabolism and volume) and/or one or more biomarkers (e.g. Aβ peptides and tau proteins (e.g. in cerebrospinal fluid (CSF)) associated with the development of a neurological disorder, such as a neurodegenerative disease, e.g. Alzheimer's disease.

Biomarkers can be objectively measured and evaluated as an indicator of disease state, prognosis, stage, risk, and treatment response. For instance, Magnetic resonance imaging (MRI) and positron emission tomography (PET) can be used to measure amyloid plaque and NFT deposition, brain metabolism and volume. Amyloid PET uses a labeled amyloid tracer, and the cortical standardized uptake ratio is calculated as an index for Aβ deposits. Certain regions of interest in the brain are determined, and the uptake is compared to a cerebellar reference. This allows objective measurement, with higher sensitivity and specificity than visual inspections of scans. Aβ₄₂ in cerebrospinal fluid (CSF) and amyloid plaque deposition in the brain are inversely correlated. Low Aβ₄₂ and high total tau (t-tau) and phosphorylated tau (p-tau) in CSF and/or high retention of amyloid tracer measured with amyloid PET in connection with AD may be used as pathophysiological markers/biomarkers as defined herein.

Biomarkers are often grouped using the ATN system, where A covers amyloid plaques, low CSF Aβ₄₂ (or low CSF Aβ₄₂/Aβ₄₀ ratio), amyloid PET and plasma. The CSF concentrations of Aβ₄₂ are reduced in patients with AD with respect to controls. This particular biomarker can provide sensitivity and specificity of the disease in 85% of cases. CSF Aβ₄₂ levels becomes abnormal in the earliest stages of AD, before amyloid PET and before neurodegeneration starts. The T covers aggregated tau, high levels of CSF p-tau, tau PET and plasma. The total concentration of tau protein in the CSF is significantly increased in patients with AD with respect to controls already in early stages of the disease. However, while it can distinguish patients and controls with sensitivity and specificity above 80%. High levels of CSF p-tau is thought to be a specific marker of AD.

The N covers neuronal injury and neurodegeneration, measured by structural MRI, PET, high CSF t-tau levels and neurofilament-light (NfL) levels.

Changes in the entorhinal cortex (EC), particularly loss of EC layer II neurons may be used as biomarker to discriminate individuals with MCI from normal control subjects.

Thus, any one or more of the biomarkers and/or physiological characteristics mentioned above may be used to identify subjects at risk of developing a neurological disorder characterized by neurodegeneration (e.g. Alzheimer's disease), such as subjects with MCI. Conveniently, the levels or amounts of the one or more biomarkers and/or the degree of physiological characteristics in the subject being assessed for treatment may be compared to a suitable control subject.

A suitable control subject may be a subject of the same sex, ethnicity and/or same age bracket as the subject being assessed, e.g. 55-59, 60-64, 65-69, 70-74 years old etc. The control subject may be characterised as having a similar general health status to the subject being assessed. The levels or amounts of the one or more biomarkers and/or the degree of physiological characteristics in the subject being assessed for treatment may be compared to predetermined values based on the assessment of a group of control subjects.

The terms “treating” or “treatment” as used herein refer broadly to any effect or step (or intervention) beneficial in the management of a clinical condition or disorder. Treatment therefore may refer to reducing, alleviating, ameliorating, slowing the development of, or eliminating one or more symptoms of the neurological disorder characterized by neurodegeneration that is being treated, relative to the symptoms prior to treatment, or in any way improving the clinical status of the subject. A treatment may include any clinical step or intervention which contributes to, or is a part of, a treatment programme or regimen.

A treatment may include delaying, limiting, reducing or preventing the onset of one or more symptoms of the neurological disorder characterized by neurodegeneration, for example relative to the symptom prior to the treatment. Thus, treatment explicitly includes both absolute prevention of occurrence or development of symptoms of the neurological disorder characterized by neurodegeneration in a subject, and any delay in the development of the neurological disorder characterized by neurodegeneration or symptom thereof, or reduction or limitation on the development or progression of the a neurological disorder characterized by neurodegeneration in a subject or symptom thereof.

As discussed further in the Examples, it is thought that the combination therapy disclosed herein attenuates the pathology associated with neurological disorders characterized by neurodegeneration, such as Alzheimer's disease-related pathology. Thus, the term “treatment” does not necessarily imply cure or complete abolition or elimination of the neurological disorder characterized by neurodegeneration or symptoms thereof.

Thus, treating the subject may be viewed as inhibiting, arresting or attenuating (i.e. slowing or reducing) the progression of cognitive impairment in a subject, e.g. a subject having or showing signs of neurodegeneration, e.g. a subject with mild cognitive impairment. Alternatively viewed, treating the subject may be inhibiting, arresting or attenuating (i.e. slowing or reducing) the pathology associated with neurological disorders characterized by neurodegeneration, such as Alzheimer's disease-related pathology.

The terms “subject”, “patient” and “individual” are used interchangeably herein and refer to a mammal, preferably a human. In particular, the terms subject, patient and individual refer to a human having a disease or disorder as defined herein in need of treatment. The subject may be at risk of developing a neurological disorder characterized by neurodegeneration, e.g. at risk of developing Alzheimer's disease. Additionally or alternatively, the subject may have mild cognitive impairment and/or one or more biomarkers and/or physiological characteristics associated with an increased risk of developing a neurological disorder characterized by neurodegeneration, such as Alzheimer's disease.

The agents disclosed herein (e.g. fasudil and lonafarnib) may be provided in pharmaceutical composition, which may be formulated according to any of the conventional methods known in the art and widely described in the literature. Thus, the agents may be formulated, separately or together, with one or more conventional carriers, diluents and/or excipients.

The agents disclosed herein may be administered systemically or locally to the subject using any suitable means and the route of administration may depend on the agent and/or the formulation of the pharmaceutical composition.

“Systemic administration” includes any form administration in which the agents (e.g. fasudil and lonafarnib) are administered to the body resulting in the whole body receiving the administered agents. Conveniently, systemic administration may be via enteral delivery (e.g. oral) or parenteral delivery (e.g. intravenous, intramuscular, subcutaneous, intratracheal, endotracheal, inhalation).

“Local administration” refers to administration of the agents at the primary site of disease (e.g. the brain, i.e. intracerebral administration) or in the local vicinity of the primary site of disease (e.g. via the fluid-filled space between the thin layers of tissue that cover the brain and spinal cord, i.e. intrathecal administration).

Reference to “systemic administration” includes intra-articular, intravenous, intraperitoneal, and subcutaneous injection, infusion, as well as administration via oral and rectal routes, or via inhalation. The agents may be administered orally.

As discussed in the Examples, the inventors have determined that local administration of the agents may provide improved availability of the agents, e.g. relative to oral administration. Thus, the agents may be administered, and may be formulated for administration, locally, e.g. intracerebrally, intrathecally or nasally. It will be evident that it may not be necessary for both agents to be administered via the same route. For instance, one agent may be administered systemically and the other locally. However, the agents both may be administered locally. Moreover, the same route of local administration may not be used for both agents, although administration via the same local route may be preferred, e.g. intracerebrally, intrathecally or nasally.

Intracerebral administration refers to administration to a specific site within the brain, e.g. an injection or infusion at a site within the brain. As the neurological disorders disclosed herein may initiate in specific parts of the brain (e.g. in the LEC layer II), it may be advantageous to target or administer the agents directly to the affected parts of the brain, i.e. the sites of disease, such as the sites of neurodegeneration, e.g. sites of Alzheimer's disease-related pathology (e.g. in the LEC layer II). Thus, any suitable form of intracerebral administration may be used in the methods and uses described herein.

In particular, intracerebral administration may be intraventricular or (focal) intraparenchymal administration, e.g. injection or infusion.

Intraventricular or intracerebroventricular administration is an invasive injection technique of substances directly into the cerebrospinal fluid in cerebral ventricles in order to bypass the blood-brain barrier.

Intraparenchymal administration refers to injection or infusion into the brain parenchyma. Focal intraparenchymal administration therefore refers to administration at a specific site of the brain parenchyma.

It will be understood that intracerebral administration may be achieved by any suitable means known in the art. As discussed in the Examples, intraventricular administration may involve implantation of a microdialysis device (e.g. probe) into the brain that is configured to enable administration of the agents directly to a cerebral ventricle. Suitable microdialysis devices (e.g. probes) are known in the art.

Intrathecal administration refers to administration to the fluid-filled space between the thin layers of tissue that cover the brain and spinal cord. This may be achieved via any suitable means known in the art.

The agents disclosed herein (e.g. fasudil and lonafarnib) can be in the form of the free drug or a pharmaceutically acceptable salt, solvate or hydrate thereof. Such salts, solvates and hydrates are well described in the art. Any suitable pharmaceutical acceptable salt, solvate or hydrate of the agents disclosed herein may be used according in the methods and treatments described herein.

The preferred forms of the agents are the forms that are present in commercial regulatory approved pharmaceutical products.

It will be evident that the agents can be administered simultaneously or in a sequence (i.e. separately). If the agents are administered in a form of a sequence, the timing between administration of the agents might vary from minutes to days depending upon the nature of the agents and the clinical situation. Moreover, as noted above, separate administration may involve administration via different routes.

Thus, the agents may be used simultaneously, separately or sequentially. When used simultaneously they are administered at the same time, but may be administered by a single route or via separate routes (e.g. a mixture administered via a single administration route or two preparations administrated at the same time but via different routes). When administered separately they may be administered at the same time or sequentially and/or may overlap in their administration timing. The agents may be administered together in a single preparation (mixture, formulation), e.g. a pharmaceutical composition comprising the agents.

The agents may be administered more than once, e.g. 2, 3, 4, 5, 6, 7, 8, 9 or 10 times (e.g. up to 20 times). This administration may be in a single (or each) cycle or in total in multiple cycles.

As referred to herein a “cycle” is a time period over which a particular treatment regime is applied and is generally repeated to provide cyclical treatment. The treatment in each cycle may be the same or different (e.g. different dosages, timings etc. may be used). A cycle may be from 7-30 days in length, e.g. a 14 day or 21 day cycle. A cycle may be about 1-3 months. Multiple cycles may be used, e.g. at least 2, 3, 4 or 5 cycles, e.g. 6, 7, 8, 9 or 10 (e.g. up to 8, 9, 10 or 20) cycles. Within each cycle the agents may be administered once or more than once, as described hereinbefore.

In view of the experimental findings discussed in the Examples, it will be understood that it may not be appropriate to maintain administration of both agents if disease progression is observed. Thus, it is contemplated that subsequent cycles of treatment may not involve the administration of both agents, e.g. administration of lonafarnib (or compounds with an equivalent function) may be stopped.

If the combined drug therapy is administered separately or sequentially, the agents (e.g. fasudil and lonafarnib) may be provided as a combined product in which the drugs are provided as separate formulations (e.g. ready for use formulations), for administration separately and/or sequentially. For instance, the combined product may comprise a kit or package containing both formulations and optionally instructions for administration.

If the combined drug therapy is administered simultaneously, the agents (e.g. fasudil and lonafarnib) may be administered together as a single drug formulation in a so-called combined preparation. Such combined preparations can easily be prepared using well-known formulation technology.

However, in some embodiments, the agents may be administered simultaneously in separate forms, e.g. separate injections or infusions.

In addition to the above-mentioned agents and combinations the compositions, kits or products disclosed herein might include other drugs. These drugs could be drugs that are known to be administered in neurological disorders.

The subject (patient) may be subjected to other treatments prior to, contemporaneously with, or after the treatments disclosed herein.

As is well known in the medical arts, doses and dosage regimens for any one subject depend upon many factors, including the subject's size, body surface area, age, agent to be administered, gender, time and route of administration, general health, stage of the disease and other drugs being administered concurrently. Accordingly, the dose and dosage regimen of the agents disclosed herein can be determined by the attending physician based on the relevant clinical factors. Thus, the agents can be administered to the subject at any suitable dose.

In a representative example, when the agents are administered intracerebrally (e.g. via intraventrical or intraparenchymal administration) or intrathecally to a human subject, the effective amount of fasudil or lonafarnib may be from about 25 μg to about 2500 μg such as about 50 μg to about 2000 μg, e.g. about 30, 40, 50, 60, 70, 80, 90, 100 μg, or about 200, 300, 400, 500, 600, 700, 800, 900, 1000, 1200, 1500 or 2000 μg.

In a further representative example, when the agents are administered orally to a human subject, the effective amount of fasudil or lonafarnib may be from about 2500 μg to about 250 mg such as about 5000 μg to about 200 mg, e.g. about 3000, 4000, 5000, 6000, 7000, 8000, 9000, 10000 μg, such as about 10, 20, 30, 40, 50, 60, 70, 80, 90, 100, 120, 150 or 200 mg.

Thus, the agents are administered in an “effective amount” or a “therapeutically effective amount”, which refers to at least an amount effective, at dosages and for periods of time necessary, to achieve the desired therapeutic or prophylactic result, i.e. at least the minimum concentration required to affect a measurable improvement of a particular disease, disorder, or condition. As noted above, an improvement may be the arrest or attenuation in the progression of cognitive impairment. An effective amount or therapeutically effective amount can be provided in one or more administrations. A therapeutically effective amount may also be an amount in which any toxic or detrimental effects of the agents or pharmaceutical compositions are outweighed by the therapeutically beneficial effects.

The patient may be subjected to other treatments prior to, contemporaneously with, or after the treatments described herein. For instance, in some embodiments, the patient may be treated with other procedures for the treatment of symptoms associated with the disease or disorder.

The agents may be provided and/or formulated for any route of administration described herein, particularly intracerebral, intrathecal, oral and administration. In particular, the agents are formulated for intraventricular (Intracerebroventricular) or intraparenchymal administration as defined herein. The skilled person ready would understand how to formulate agents disclosed herein for these routes of administration.

Examples of suitable pharmaceutical carriers, excipients and/or diluents are well-known in the art and include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, or mixtures thereof.

Pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Examples of oils are those of animal, vegetable, or synthetic origin, for example, peanut oil, soybean oil, olive oil, sunflower oil, fish-liver oil, another marine oil, or a lipid from milk or eggs.

Aqueous carriers include water, alcoholic/aqueous solutions, emulsions or suspensions, including saline and buffered media such as phosphate buffered saline solutions, water, emulsions, such as oil/water emulsions, various types of wetting agents, sterile solutions etc. Compositions comprising such carriers can be formulated by well-known conventional methods. Suitable carriers may comprise any material which, when combined with the agents, retains the biological activity.

The pharmaceutical composition may be in a “ready to use” formulation that contains the agent(s) in dissolved or solubilized form and is intended to be used as such or upon further dilution in pharmaceutically acceptable diluents. However, the pharmaceutical composition may be provided in a solid form, e.g. as a lyophilizate, to be dissolved in a suitable solvent to provide a liquid formulation.

As discussed above, there is a desire for robust preclinical models of neurological disorders with improved comparability with the human conditions, which enable the assessment of drug candidates and their suitability for translation into the clinic. To this end, the inventors have developed systems and methods for assessing the effect of one or more candidate drugs in a neurological disorder.

Accordingly, provided herein is a system for assessing the effect of one or more candidate drugs in a neurological disorder animal model comprising:
(a) a transgenic animal model having physiological characteristics associated with a neurological disorder characterized by neurodegeneration;
(b) means for administering said one or more candidate drugs intracerebroventricularly to the transgenic animal model; and optionally
(c) means for obtaining a sample from the transgenic animal model for analysis of one or more biomarkers associated with a neurological disorder characterized by neurodegeneration;
wherein the transgenic animal model has been additionally modified to express exogenous tau protein in neurons.

Also provided herein is a method for assessing the effect of one or more candidate drugs in a neurological disorder animal model comprising:
(a) providing a transgenic animal model having physiological characteristics associated with a neurological disorder characterized by neurodegeneration;
(b) modifying the transgenic animal model of (a) to express exogenous tau protein in neurons;
(c) administering said one or more candidate drugs intracerebroventricularly to the transgenic animal model; and
(d) analysing one or more biomarkers associated with a neurodegenerative disease in a sample obtained from the transgenic animal and/or assessing the cognitive ability and/or behaviour of the transgenic animal, thereby assessing the effect of the one or more candidate drugs.

A transgenic animal model having physiological characteristics associated with a neurological disorder characterized by neurodegeneration may have any one or more physiological characteristics associated with the neurological disorders described herein, such as amyloid plaques, NFTs and/or cognitive impairment.

The transgenic animal model may be a murine animal model, particularly a mouse animal model. The transgenic animal model may comprise more than one genetic modification that results in the one or more physiological characteristics associated with the neurological disorders described herein. In particular, the transgenic animal (e.g. mouse) model: (a) overexpresses an APP gene comprising the Swedish APP gene mutation (APP_{SWE}); (b) overexpresses a microtubule associated protein tau (MAPT) comprising the P301L gene mutation (MAPT_{P301L}); and (c) comprises the mutant M146V in the Presenilin-1 (PSEN1) gene (PSEN1_{M146V}). Thus, the transgenic animal model may be the 3xTg mouse model, which is based on a C57BL/6; 129X1/SvJ; 129S1/Sv background and contains the three human gene mutations specified above.

Advantageously, the inventors have determined that the 3xTg mouse model may be improved by further modifying the animal to express exogenous tau protein in neurons, e.g. neurons in the LEC layer II. It will be understood that exogenous expression of tau protein may be achieved by any suitable means known in the art. In a representative example, expression may be induced by administration (e.g. injection) of a vector (e.g. a viral vector) encoding a tau protein into the target site, i.e. the LEC layer II.

The vector must be capable of transfecting or transducing a cell (e.g. neuron), such that it expresses the tau protein.

The vector may be a non-viral vector such as a plasmid. Plasmids may be introduced into cells using any well-known method of the art, e.g. liposomes, or cell penetrating peptides (e.g. amphipathic cell penetrating peptides).

The vector may be a viral vector, such as a retroviral, e.g. a lentiviral vector or a gamma retroviral vector, or adeno-associated viral vector.

Vectors suitable for delivering nucleic acids for expression in mammalian cells are well-known in the art and any such vector may be used. Vectors may comprise one or more regulatory elements, e.g. a promoter, such as the chicken beta actin (CBA) promoter.

Delivery systems are also available in the art which do not rely on vectors to introduce a nucleic acid molecules into a cell, for example, systems based on transposons, CRISPR/TALEN delivery and mRNA delivery. Any such system can be used to deliver a nucleic acid molecule according to the present invention.

As discussed in the Examples, it may be useful or necessary to express more than one polypeptide in the cell, e.g. a reporter polypeptide such as GFP in addition to the tau protein. Thus, the vector may comprise a nucleic acid encoding a first polypeptide (e.g. GFP) and a nucleic acid encoding a tau polypeptide. The vector may comprise the nucleic acid molecules as separate entities, or as a single nucleotide sequence. If they are present as a single nucleotide sequence, they may comprise one or more internal ribosome entry site (IRES) sequences or other translational coupling sequences between the two encoding portions to enable the downstream sequence to be translated. A cleavage site such as a 2A cleavage site (e.g. T2A, F2A or P2A) may be encoded by a nucleic acid. Alternatively, the nucleic acid encoding a first polypeptide (e.g. GFP) and the nucleic acid encoding the tau polypeptide may be introduced to a cell as separate entities, e.g. on different vectors.

A tau protein typically refers to one of six highly soluble protein isoforms produced by alternative splicing from the gene MAPT (microtubule-associated protein tau). Any suitable tau protein may be used to modify the transgenic animal model described herein. In particular, the tau protein is a human tau protein or mutant thereof. In particular, the mutant tau protein may comprise the P301 L mutation. Thus, the tau protein may comprise an amino acid sequence as set forth in any of the following Uniprot accessions: P10636, P18518, Q14799, Q15549, Q15550, Q15551, Q1RMF6, Q53YB1, Q5CZ17, Q5XWF0, Q6QT54, Q9UDJ3, Q9UMH0 and Q9UQ96, optionally wherein the tau protein comprises the P301 L mutation.

The drug candidates may be administered intracerebroventricularly to the transgenic animals using any suitable means known in the art. In a representative embodiment, the means for intracerebroventricular administration comprises a microdialysis device or probe (e.g. a β-irrigated 2 mDa microdialysis probe) which is implanted into the brain of the transgenic animal and configured to enable administration of the agents directly to a cerebral ventricle, e.g. via injection. Advantageously, the microdialysis device (e.g. probe) also provides the means for obtaining a sample (e.g. CSF) for analysis of one or more biomarkers associated with a neurological disorder characterized by neurodegeneration. In this respect, the system/device/probe is configured to enable administration of said one or more candidate drugs and obtaining a sample for analysis simultaneously or contemporaneously, i.e. to provide so-called push-pull microdialysis. Thus, the microdialysis device (e.g. probe) may comprise an inlet and outlet configured to enable simultaneous administration of drug candidates and collection of a biological sample, e.g. CSF. The inlet and outlet may be connected to a device for delivery of the drug candidates (e.g. a syringe) and a device for collection of the biological sample (e.g. peristaltic pump and sample or fraction collector), respectively. The connections may be provided by suitable tubing, such as peristaltic tubing, particularly fluorinated ethylene propylene (FEP) peristaltic tubing. A representative configuration is described in Bjorkli et al., 2021 (J. Alzheimers Dis. 84 (4), pp. 1781-1794, which is herein incorporated by reference).

The step of analysing one or more biomarkers associated with a neurodegenerative disease in a sample (e.g. CSF) obtained from the transgenic animal may be achieved by any suitable means. For instance, the sample may be cerebrospinal fluid (CSF) and analysis may involve proteomic analysis of the fluid, e.g. analysing the concentration of particular proteins, such as amyloid-β and/or tau proteins. Proteomic analysis may be achieved using any suitable means known in the art, e.g. ELISA. Analysis may involve harvesting tissue from the transgenic animal (e.g. brain tissue) and performing immunohistochemistry and/or microscopy analysis, e.g. quantification of intraneuronal amyloid-β and/or and tau, and/or amyloid plaques.

The step of assessing the cognitive ability and/or behaviour of the transgenic animal may be achieved using any suitable tests known in the art, e.g. context-dependent spatial memory testing. Representative tests are described in the Examples.

It will be understood that candidate drugs refer to any substance that may induce a pharmacological and/or physiological effect. The term also encompasses pharmaceutically acceptable and pharmacologically active forms thereof, including salts. Thus, a candidate drug may be a proteinaceous, non-proteinaceous (e.g. chemical entity) or nucleic acid molecule.

Proteinaceous molecules include peptides, polypeptides and proteins. The terms polypeptide and protein are used interchangeably herein.

Non-proteinaceous molecules include small, intermediate or large chemical molecules as well as molecules identified from natural product screening or the screening of chemical libraries. Natural product screening includes the screening of extracts or samples from any suitable source of natural products including plants, microorganisms, soil, river beds, coral and aquatic environments for molecules or groups of molecules which may induce a pharmacological and/or physiological effect.

Nucleic acid molecule drugs (“nucleic acids” or “polynucleotides”) include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art. Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule.

Preferred aspects of the methods, uses and products defined herein are as set out in the Examples in which one or more of the parameters or components used in the Examples may be used as preferred features of the methods, uses and products described hereinbefore.

The invention will now be described in more detail in the following non-limiting Examples with reference to the following drawings in which:

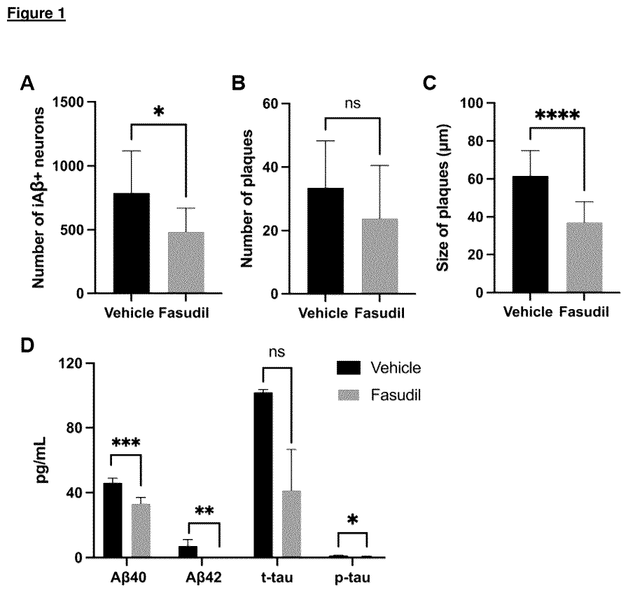

FIG. 1 shows that the inhibition of the Wnt-PCP pathway attenuated amyloid-β and tau pathology in early and late phase AD. Specifically, FIG. 1 shows bar charts depicting: (A) Mean number of Aβ+ neurons in dSub of 3xTg AD mice after infusions of a vehicle and Fasudil. Intraneuronal Aβ in dSub was quantified from at least seven brain sections for each animal using Ilastik. Error bars denote ±1 SD, unpaired two-tailed t-test, *p<0.05; (B) Mean number of amyloid plaques in dSub of 3xTg AD mice after infusions of a vehicle and Fasudil. Amyloid plaques in dSub were quantified from at least four brain sections for each animal using Ilastik. Error bars denote ±1 SD, unpaired two-tailed t-test, n. s.: non-significant; (C) Mean size of amyloid plaques in dSub of 3xTg AD mice after infusions of a vehicle and Fasudil. Amyloid plaques in dSub were quantified from at least 4 brain sections for each animal using Ilastik. Error bars denote ±1 SD, unpaired two-tailed t-test, ****: p<0.0001; (D) Mean concentrations of duplicates of Aβ40, Aβ42, p-tau and t-tau CSF levels (pg/ml) as measured by multiplex ELISA on the last day of infusion (day 14) of a vehicle (n=6) or Fasudil (n=10) in young and older 3xTg AD mice. Error bars denote ±1 SD, unpaired two-tailed t-test, ***: p<0.001, **: p<0.01, *: p<0.05.

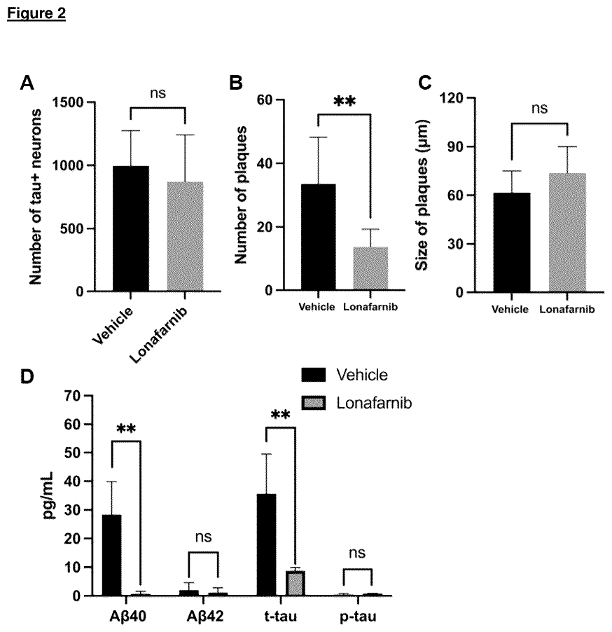

FIG. 2 shows that the induction of autophagy attenuated amyloid-β and tau pathology in early and late phase AD. Specifically, FIG. 2 shows bar charts depicting: (A) Mean number of tau+neurons in LEC of 3xTg AD mice after infusions of a vehicle and Lonafarnib. Intraneuronal tau in LEC layer II was quantified from at least four brain sections for each animal using Ilastik. Error bars denote ±1 SD, unpaired two-tailed t-test, n.s.: non-significant; (B) Mean number of amyloid plaques in dSub of 3xTg AD mice after infusions of a vehicle and Lonafarnib. Amyloid plaques in dSub were quantified from at least 4 brain sections for each animal using Ilastik. Error bars denote ±1 SD, unpaired two-tailed t-test, **: p<0.01; (C) Mean size of amyloid plaques in dSub of 3xTg AD mice after infusions of a vehicle and Lonafarnib. Amyloid plaques in dSub were quantified from at least four brain sections for each animal using Ilastik. Error bars denote ±1 SD, unpaired two-tailed t-test, n.s.: non-significant; (D) Mean concentrations of duplicates of Aβ40, Aβ42, p-tau and t-tau CSF levels (pg/ml) as measured by multiplex ELISA on the last day of infusion (day 10) of a vehicle (n=8) or Lonafarnib (n=4) in young (injected with AAV-tau) and older 3xTg AD mice. Error bars denote ±1 SD, unpaired two-tailed t-test, **: p<0.01.

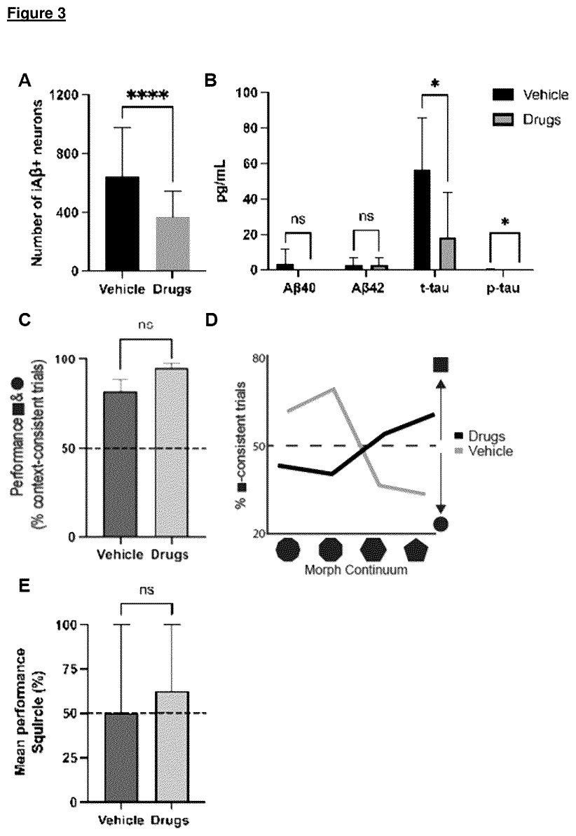

FIG. 3 shows that combinatorial targeting of Wnt and mTOR pathways effectively attenuates neuropathology and context-dependent spatial memory deficits. Specifically, FIG. 3 shows bar charts and line plots depicting: (A) Mean number of Aβ+ neurons in dSub of 3xTg AD mice after infusions of a vehicle, or Fasudil and Lonafarnib. Intraneuronal Aβ in dSub was quantified from at least seven brain sections for each animal using Ilastik. Error bars denote ±1 SD, unpaired two-tailed t-test, ****p<0.0001; (B) Mean concentrations of duplicates of Aβ40, Aβ42, p-tau and t-tau CSF levels (pg/ml) as measured by multiplex ELISA on the last day of infusion (day 7) of a vehicle (n=6) or Fasudil and Lonafarnib (n=4) in young 3xTg AD mice. Error bars denote ±1 SD, unpaired two-tailed t-test, *p<0.05; (C) Mean behavioral performance (% of trials with context-appropriate choices) in 3xTg AD mice infused with a vehicle (n=3) or Fasudil and Lonafarnib (n=4) in the square- and circle-chambers. Error bars denote ±1 SD, unpaired two-tailed t-test, n.s.: non-significant; (D) Percentage of trials with square context-consistent digs in 3xTg AD mice infused with a vehicle (n=3) or Fasudil and Lonafarnib (n=4) in each of the four morphed environments, ranging from most circle-like to most square-like in boundary geometry shape; (E) Mean behavioral performance (% of trials with context-appropriate choices) in 3xTg AD mice infused with a vehicle (n=3) or Fasudil and Lonafarnib (n=4) in the Squircle context. Error bars denote ±1 SD, unpaired two-tailed t-test, n.s.: non-significant.

Abbreviations: AAV, adeno-associated virus; AD, Alzheimer's disease; Aβ, amyloid-β; CSF, cerebrospinal fluid; dSub, dorsal subiculum; ELISA, enzyme-linked immunosorbent assay; iAβ, intraneuronal Aβ; LEC, lateral entorhinal cortex; SD, standard deviation; t-tau, total tau; p-tau, phosphorylated tau; Wnt-PCP, Wnt-planar cell polarity.

### EXAMPLES

### Materials and Methods

Animals

Thirty 3xTg AD mice (MMRRC Strain #034830-JAX; RID:MMRRC_034830-MU) and two control B6129 mice (Strain #:101045; RRID: IMSR_JAX:101045) were included in these experiments. 3xTg AD mice contain three mutations associated with familial Alzheimer's disease (APP_{swe}, MAPT_{P301L}, and PSEN1_{M146V}). The 3xTg AD mouse model male transgenic mice may not exhibit all phenotypic traits of AD. Therefore, only female mice were included in these experiments.

To validate whether our mouse model replicated AD neuropathology as observed in patients, and to assess the possibility of genetic drift in our own colony, we characterized the 3xTg AD mouse model. Our findings suggest that genetic drift with phenotypic effects occurred in our mouse colony, but we also confirm that this mouse presents as a valid model for studying AD-related neuropathology. All housing and breeding of animals was approved by the Norwegian Animal Research Authority and is in accordance with the Norwegian Animal Welfare Act §§ 1-28, the Norwegian Regulations of Animal Research §§ 1-26, and the European Convention for the Protection of Vertebrate Animals used for Experimental and Other Scientific Purposes (FOTS ID 21061). The animals were kept on a 12 h light/dark cycle under standard laboratory conditions (19-22° C., 50%-60% humidity), and had free access to food and water.

Microdialysis Guide Implantation Surgeries

Surgeries were based on previously established protocols (Bjorkli et al., 2021, supra). Implantation surgery was performed to insert microdialysis guide cannulas (CMA 7; CMA Microdialysis AB, Kista, Sweden) into the lateral ventricle of mice. Mice were anesthetized with isoflurane gas (4% induction and 1.5-3% maintenance; IsoFlo vet., Abbott Laboratories, Chicago, IL, United States) prior to being fixed in a stereotaxic frame (Kopf Instruments; Chicago, IL, United States). Prior to making any incisions, Marcain (0.03-0.18 mg/kg; Aspen Pharma, Ballerup, Denmark) was injected subcutaneously into the scalp and Metacam (5 mg/kg; Boehringer Ingelheim Vetmedia, Copenhagen, Denmark) and Temgesic (0.05-0.1 mg/kg; Invidor United Kingdom, Slough, Great Britain) were administered subcutaneously for intraoperative pain relief. Equal heights of bregma and lambda were measured to ensure that the skull was level for each animal (with ±0.1 mm tolerance), as well as two points equally distant from the midline. After leveling the skull, the stereotaxic coordinates were derived to target the lateral ventricle (A/P: −0.1 mm, M/L: +1.2 mm, D/V: −2.75 mm). The microdialysis guide cannula was attached to the stereotaxic frame using a guide clip and connection rod for the clip (CMA Microdialysis AB, Kista, Sweden). The skull was drilled through at these coordinates and the guide cannula was slowly lowered into the drilled hole. The guide cannula was attached to the skull with super glue and dental cement (Dentalon Plus; Cliniclands AB, Trelleborg, Sweden). Post-surgery, Metacam and Temgesic were administered within 24 h. The guide cannula was implanted into the right hemisphere of all animals, as we did not observe any lateralization of pathology in the brains of 3xTg AD mice.

Stereotactic Viral Injections of P301L Tau

Mice were treated identically to microdialysis guide implantation surgeries, up until deriving stereotaxic coordinates. To target LEC layer II, a craniotomy was made at 0.5 mm anterior to lambda and ˜4 mm lateral (dependent on animal weight) to the midline. A Hamilton microsyringe (Neuros 32-gauge syringe, 5 μl, Hamilton company, Nevada, United States) was lowered vertically into the brain to a depth ˜3.6 mm (dependent on animal weight) from the surface, and 300-1,500 nl of viruses was injected using a microinjector (Nanoliter 2010, World Precision Instruments Inc., United States). We injected the adeno-associated virus (AAV)8 GFP-2a-P301 Ltau (with the chicken beta actin [CBA] promoter; hereafter referred to as AAV-tau). In AAV-tau the short 2A peptide cleaves GFP and human tau during translation at the ribosome. This results in neurons transduced with the virus being able to produce GFP and human tau as individual proteins (GFP+/MC1+; donor neurons). Conversely, neurons that receive human tau from cross-neuronal spread have human tau, but no GFP (GFP−/MC1+; recipient neurons). The microsyringe was kept in place for 5 min prior and after the injection, to minimize potential upward leakage of the viral solution. Metacam was given within 24 h post-surgery.

Animals were implanted with microdialysis guide cannulas 2 months following injections. Push-pull microdialysis apparatus and sampling Push-pull microdialysis was conducted as previously described (Bjorkli et al., 2021 supra). A refrigerated fraction collector (CMA 470) was set to 6° C. for the storage of collected CSF in 300 μl low-retention polypropylene plastic vials (Harvard Apparatus, Cambridge, MA, United States). Fluorinated ethylene propylene (FEP) peristaltic tubing (CMA Microdialysis AB, Kista, Sweden) was placed inside each plastic vial for collection and connected to the cassette of the peristaltic roller pump (Reglo ICC Digital). This peristaltic FEP tubing was connected to the outlet side of microdialysis probes (β-irrigated 2 mDa microdialysis probe; CMA 7; CMA Microdialysis AB, Kista, Sweden) with a polyethersulfone 2 mm membrane with tubing adapters bathed in 75% ethanol. FEP tubing (CMA Microdialysis AB, Kista, Sweden) was connected to each microsyringe. The FEP tubing was then connected to the inlet part of the microdialysis probes. Transparent cages were prepared with 1.5 cm of bedding, filled water bottles, and treats. Saline or drugs were loaded inside a gastight microsyringe (CMA Microdialysis AB, Kista, Sweden), which was placed into a syringe pump (CMA 4004). The “dead volume” of the FEP outlet tubing (1.2 ml/100 mm) was calculated. 100 cm of FEP outlet tubing was used, and therefore the first 12 ml sampled from each animal were discarded. Prior to inserting the microdialysis probes into the guide cannula, the probe was conditioned in 75% ethanol for better recovery of analytes. At the conclusion of microdialyte sampling, the vials of 60 μl CSF were centrifuged and kept at −80° C. until the samples were analyzed with multiplex ELISA.

Intraventricular Drug Infusions

Fasudil and Lonafarnib have previously been delivered using DMSO, which can damage the BBB and mitochondria as well as cause apoptosis. Since we had a less effective delivery vehicle than DMSO, we conducted pilot experiments to determine effective titers of Fasudil and Lonafarnib, as well as to determine the most effective duration of infusions. Previous research has shown that ˜98% of all small molecules are not transported across the BBB, whilst other research has shown poor drug transport from CSF to the brain. Taking drug transport across the BBB, and from CSF into the brain parenchyma into consideration, dosages of both 25, 50 and 80 mg/kg were administered in initial pilot experiments.

In these experiments, a final concentration of 50 mg/kg of Fasudil (10 mM; Selleck Chemicals, Houston, TX, United States) was infused for 14 days in mice and stored at −80° C., whilst a final concentration of 80 mg/kg of Lonafarnib (5 mM; Cayman Chemical, Ann Arbor, MI, United States) was infused for 10 days and stored −20° C. between infusions. These drug concentrations resulted in no observable side-effects in mice. The same dosages were used during combinatorial infusions of Fasudil and Lonafarnib (administered for a duration of 7 days) in mice (n=4) as the drugs target independent intracellular pathways. All dosages in ml were calculated using; dosage (mg)/concentration (mg/ml)=dose×ml) and were infused at a volume of 60 μl at a rate of 1 μl/min using saline as a control vehicle. To assess the efficacy of oral versus intraventricular drug administration, we mixed 0.6 ml of 50 mg/kg Fasudil and 80 mg/kg Lonafarnib in baby porridge (Nestlé S.A., Vevey, Switzerland) in 3xTg AD mice (n=3). Intraventricular drug infusions were more effective in reducing intraneuronal Aβ accumulation in the dorsal subiculum (dSub) compared to oral administration of the drugs (t65=2.54, p=0.0136, unpaired two-tailed t-test).

We immunolabelled for lysosomal associated membrane protein 1 (LAMP1) in 3xTg AD mice. LAMP1+ neurons in dSub were more prominent in Lonafarnib infused (n=4), compared to vehicle infused mice (n=8), and overlapped with fibrillar OC+amyloid plaques.

Context-Dependent Spatial Memory Testing

The basic training and testing protocol of the context dependent spatial memory task involved the following: starting 5 days before the experiment, animals were taught to dig in a brain cup for a food reward (Weetos choco, Nestlé S.A.) in their home cage by providing them once daily with the reward gradually buried deeper under ginger-scented bedding (1 g of ginger for every 100 g of bedding) while being gradually food deprived to maintain 90-95% of their free-feed weight.

Disoriented mice were trained to dig for buried food rewards in two different chambers, one with square boundaries (4×29.25 cm) and one with circle boundaries (157 cm circumference). All chambers were built out of rectangular Legos (2×1 cm; Lego A/S, Billund, Denmark), and were 15 cm tall. Rewards were buried under ginger-scented bedding in cups embedded in the chamber floors. Each chamber was surrounded by the same distal cues for orientation. There were four possible reward locations in each chamber, and the rewarded location differed between the square- and circle chamber relative to the common reference frame provided by the distal cues. Pilot experiments revealed that mice could successfully discriminate the square and circle reward locations above chance after 8 trials. Therefore, the training phase consisted of four training trials per chamber per day for 2 days, with successive trials alternated across chambers (8 trials total in the square-chamber and 8 trials total in the circle chamber).

If a given mouse achieved 66.6% correct performance during training, contextual memory was then tested in 4 testing sessions across 4 days, with 8 trials per session. During each testing session, the first two trials consisted of spatial memory being tested in the square- and circle-chamber with rewards. In trials 3-6, spatial memory was tested in four chambers with morphed boundary geometry, which continuously ranged from most-square-like to most-circle-like: a pentagon (5×31.4 cm), a hexagon (6×26.16 cm), an octagon (8×19.6 cm), and a decagon (10×15.7 cm). During the final two trials of each testing session (trials 7-8), the animals were again tested in the square- and circle-chambers with rewards. The order of the square-, circle-, and morphed-chambers across trials in each session was randomized but was the same for each animal on a given day's session. If a given mouse achieved 66.6% correct performance during testing, contextual memory was tested in an ambiguous half-square half-circle context (the “Squircle”).

During reward trials, mice were removed from the apparatus and the trial ended after they had found the reward. During unrewarded trials, they were removed, and the trial ended after their first dig, or after 5 min (whichever came later). Chambers were cleaned with ethanol after each trial to remove odor trails. Dig locations and time spent in these locations were calculated using ANY-maze video tracking system (Stoelting Europe) via an overhead, centrally located camera (DMK 22AUC03 USB 2.0 monochrome industrial camera, The imaging Source Europe, Germany).

Proteomic Analysis of Amyloid-β and Tau Concentrations in Cerebrospinal Fluid

The MILLIPLEX® MAP human Aβ and tau magnetic bead panel 4-plex ELISA kit (Millipore, Burlington, MA, United States) and the Bio-Plex 200 System instrument (Biorad, Hercules, CA, United States) were used to assess simultaneously the concentrations of Aβ40, Aβ42, total tau (t-tau), and phosphorylated tau at Thr181 (p-tau) in CSF samples. The samples were undiluted and analyzed in duplicates.

Tissue Processing and Immunohistochemistry

Mice were administered a lethal dose of sodium pentobarbital (100 mg/ml; Apotekforeningen, Oslo, Norway) and transcardially perfused with Ringer's solution followed by paraformaldehyde (PFA, 4%; Sigma-Aldrich) in 125 mM phosphate buffer (PB). Brains were extracted and fixed for a minimum of 24 h in PFA at 4° C. and transferred to a 2% DMSO solution prepared in PB for 24 h at 4° C. Brains were sectioned coronally at 40 μm on a freezing-sliding microtome (Microm HM430, ThermoFisher Scientific, Waltham, MA, United States).

An incision was made in the non-implanted hemisphere for visualization of the control hemisphere. Immunohistochemical processing was conducted on the tissue. Previous research has indicated that a differential microtubule-associated protein 2 (MAP2) immunolabeling pattern can distinguish dense-core from diffuse amyloid plaques using DAB (Sigma-Aldrich, St. Louis, MO, United States) as a chromogen.

One series of each brain was dehydrated in ethanol, cleared in xylene (Merck Chemicals, Darmstadt, Germany) and rehydrated before staining with Cresyl violet (Nissl; 1 g/L) for 3 min to verify probe placement. The sections were then alternatively dipped in ethanol-acetic acid (5 ml acetic acid in 1 L 70% ethanol) and rinsed with cold water until the desired differentiation was obtained, then dehydrated, cleared in xylene and cover-slipped with entellan containing xylene (Merck Chemicals). Sections were scanned using a Mirax-midi slide scanner (objective 20X, NA 0.8; Carl Zeiss Microscopy, Oberkochen, Germany), using either reflected fluorescence (for sections stained with a fluorophore) or transmitted white light (for sections immunolabelled with NissIDAB, or Gallyas-silver) as the light source.

Quantification of Intraneuronal Amyloid-β and Tau, and Amyloid Plaques

Series of sections were chosen randomly and coded to ensure blinding to the investigators. The number of cells containing intraneuronal Aβ, tau, and amyloid plaques, in dSub and LEC of 3xTg AD mice infused with a vehicle or drugs was estimated with Ilastik using the Density Cell Counting workflow.

dSub and LEC was delineated using cytoarchitectonic features in sections stained with Nissl, based on The Paxinos & FranklinMouse Brain Atlas. The same surface area and rostrocaudal levels of each brain region was selected, and at least 4 brain sections were used for each infused hemisphere. Damaged regions of brain sections were excluded from analyses to avoid false-positive antibody expression.

Statistics

Effect size (Cohen's D) was calculated based on initial experiments between animals infused with Fasudil and animals infused with a vehicle, and the resulting effects on intraneuronal Aβ in dSub. Based on each group consisting of n=2 animals, an effect size of 0.75 was calculated (0.8 is considered a large effect size). Most of the dataset was normally distributed (Shapiro-Wilk test) and therefore two-tailed, unpaired t-tests were used to compare mean differences. For the minor parts of the dataset that was not normally distributed, nonparametric statistical tests were used (Mann-Whitney U). Statistical comparisons of behavioral data across vehicle and drug infused mice were conducted based on trial-wise pooling of data across mice separately for each group. Behavioral performance in the morphed environments of the context-dependent spatial memory task was calculated as follows: dig in square-consistent location=1, dig in circle-consistent location=0, dig in any other location=0.5. Context-consistency of reward locations was determined relative to the common reference frame defined by the distal cues shared across all contexts. We then assessed whether performance in the morphed environments was associated with more context-appropriate choices for animals treated with a vehicle compared to drugs. All statistical tests and graphs were made in Prism 9 (GraphPad Software Inc., CA, United States).

### Example 1: Fasudil Treatment Attenuates Amyloid-β and Tau Patholoqy in Early and Late Phases of the Disease

First, we wanted to assess whether Fasudil (administered for a duration of 14 days) would affect intraneuronal Aβ which is present already at 1-month-of-age in 3xTg AD mice.

Intraventricular administration of Fasudil in 6 months-old 3xTg AD mice reduced the number of Aβ+ neurons in dSub as compared to vehicle infused mice (t18=2.63, p=0.0169, unpaired two-tailed t-test; FIG. 1A). We then went on to assess the effect of Fasudil on amyloid plaques in 3xTg AD mice, which accumulate at 13-months-of-age. In older 3xTg AD mice (14 months-old), Fasudil infusions moderately reduced the number of dense-core amyloid plaques in dSub as compared to vehicle infused mice (n.s.; FIG. 1B). The size of amyloid plaques, on the other hand, was effectively reduced in Fasudil, compared to vehicle, treated mice (t26=4.69, p<0.0001, unpaired two-tailed t-test; FIG. 1C). We went on to conduct proteomic analyses to assess CSF Aβ and tau levels following Fasudil treatment (FIG. 1D).

On average across younger and older mice, Fasudil effectively reduced CSF Aβ40 (t6=5.96, p<0.001, unpaired two-tailed t-test) and Aβ42 levels (t5=5.59, p<0.01, unpaired two-tailed t-test). Fasudil treatment also effectively reduced CSF p-tau levels (t5=2.86, p<0.05, unpaired two-tailed t-test), and moderately reduced CSF t-tau levels (n.s.). Lonafarnib treatment attenuated amyloid-β and tau pathology in early and late phases of the disease.

Similarly, we also wanted to assess whether Lonafarnib (administered for a duration of 10 days) would affect AD-related neuropathology present in early phases of AD. Since previous research suggests that Lonafarnib can reduce tau levels, we assessed not only early intraneuronal Aβ but also early tau abnormalities following infusions. Lonafarnib did not affect the number of Aβ+ neurons in dSub in young 3xTg AD mice. Since the earliest accumulation of non-fibrillar tau (recognized by the MC1 antibody) is not present in 3xTg AD mice until around 9-months-of-age in CA1, we overexpressed human tau in LEC layer II (an area that is early involved in NFT deposition in AD patients), of 6 months-old 3xTg AD mice. Lonafarnib infusions moderately reduced the number of tau+ neurons in LEC following injection of AAV-tau (n.s.; FIG. 2A).

In older 3xTg AD mice (14 months-old), Lonafarnib infusions effectively reduced the number of dense-core amyloid plaques that overlapped with MC1 immunolabelling in dSub as compared to vehicle infused mice (t14=3.86, p=0.0017, unpaired two-tailed t-test; FIG. 2B). The size of amyloid plaques, on the other hand, was moderately increased in Lonafarnib, compared to vehicle, treated mice (n.s.; FIG. 2C). We then conducted proteomic analyses to assess CSF As and tau levels following Lonafarnib treatment (FIG. 2D).

On average, across younger (injected with AAV-tau) and older mice, Lonafarnib effectively reduced CSF Aβ40 (t6=4.53, p<0.01, unpaired two-tailed t-test) and t-tau levels (t6=4.05, p<0.01, unpaired two-tailed t-test). Lonafarnib treatment moderately reduced CSF AB42 (n.s.) and p-tau levels (n.s.).

### Example 2: Combinatorial Drug Treatment Effectively Attenuates Alzheimer's Disease-Related Pathology at the Molecular and Functional Level

Since both drugs appeared to reduce AD-related neuropathology, we wanted to assess their combinatorial effects when administered for 7 days during earlier phases of the disease (4-months-of-age). Since these mice were young, the treatment effects on intraneuronal Aβ in dSub were assessed, without the possibility of assessing amyloid plaques or tau pathology.

Combinatorial treatment of drugs effectively reduced the number of Aβ+neurons in dSub of mice (t80=4.66, p<0.0001, unpaired two-tailed t-test; FIG. 3A).

Proteomic analyses of CSF Aβ and tau levels (FIG. 3B) revealed that on average, combinatorial treatment effectively reduced CSF t-tau (t10=2.39, p<0.05, unpaired two-tailed t-test) and p-tau levels (t10=2.24, p<0.05, unpaired two-tailed t-test). Combinatorial treatment moderately reduced CSF Aβ40-42 levels (n.s.).

We then examined context-dependent spatial memory in 3xTg AD mice at 4-months-of-age, the same age as when cognitive deficits usually begin in this model, after vehicle and drug infusions.

Mice were trained to search for buried food rewards in two different contexts, one square and one circle. Animals infused with drugs initially searched in context-appropriate reward locations slightly more often than those infused with a vehicle. (n.s.; FIG. 3C). We further examined contextual memory recall in environments with progressive morphs of boundary geometry, increasing the number of boundary walls from most square-like to most circle-like. Compared to animals infused with a vehicle, drug infused mice searched more often in context-consistent reward locations (X²=69.17, p<0.0001; Pearson's Chi-squared test; FIG. 3D). Furthermore, when contextual memory was tested in an ambiguous half-square half-circle context (the “Squircle”), mice infused with drugs were slightly more likely to dig at a location previously associated with either the square or circle, compared to locations not associated with any context, and compared to control animals infused with a vehicle (n.s.; FIG. 3E). Thus, combinatorial drug treatment led to an improvement of cognitive deficits usually associated with canonical AD.

### SUMMARY

Treatment with Fasudil reduced early intraneuronal Aβ, the number and size of amyloid plaques in dSub, and CSF Aβ40-42 and p-tau levels. Lonafarnib infusions, on the other hand, did not affect intraneuronal Aβ but rather reduced early non-fibrillar forms of tau after overexpression in LEC layer II. Treatment with Lonafarnib also reduced the number of amyloid plaques, but unexpectedly increased their size in dSub, and only effectively decreased CSF Aβ40 and t-tau levels. Both drugs affected dense-core, rather than diffuse, amyloid plaques, and the former is associated with microglial activation, neurodegeneration, and cognitive decline in AD patients.

We found that novel combinatorial administration of these drugs effectively reduced early intraneuronal Aβ in younger mice, led to reduced CSF Aβ40 and p- and t-tau levels, and improved context-dependent spatial memory.

Thus, combinatorial treatment with both drugs was effective at reducing intraneuronal Aβ and led to improved cognitive performance in mice. Overexpression of tau in LEC layer II was effectively reduced by Lonafarnib at early stages, but treatment led to an increase in size of amyloid plaques at later stages of AD. Combinatorial treatment of both drugs when 3xTg AD mice start to display cognitive impairments not only reduced intraneuronal Aβ, but also attenuated context-dependent spatial memory deficits.

1. A method of treating or preventing a neurological disorder characterized by neurodegeneration in a subject, the method comprising administering to the subject a therapeutically effective amount of:
(i) a Rho-associated protein kinase (ROCK) inhibitor or a pharmaceutically acceptable salt thereof; and
(ii) a farnesyltransferase inhibitor or a pharmaceutically acceptable salt thereof.

2. The method of claim 1, wherein the ROCK inhibitor or a pharmaceutically acceptable salt thereof is fasudil or a pharmaceutically acceptable salt thereof.

3. The method of claim 1, wherein the farnesyltransferase inhibitor or a pharmaceutically acceptable salt thereof is lonafarnib or a pharmaceutically acceptable salt thereof.

4. The method of claim 1, wherein the method comprises administering (i) and (ii) separately, simultaneously or sequentially.

5. The method of claim 1, wherein the method comprises administering (i) and/or (ii) intracerebrally, intrathecally or nasally.

6. The method of claim 5, wherein administering intracerebrally comprises intracerebroventricular administration or focal intraparenchymal administration.

7. The method of claim 1, wherein (i) and (ii) are provided in a pharmaceutical composition together with a pharmacologically acceptable excipient and/or diluent.

8. The method of claim 1, wherein the neurological disorder characterized by neurodegeneration is a neurodegenerative disease, a neurological disorder that affects memory, or a temporal lobe memory disorder.

9. The method of claim 1, wherein the neurological disorder characterized by neurodegeneration is selected from: dementia, which includes frontotemporal dementia or frontotemporal degeneration, vascular dementia, mixed dementia, dementia with Lewy bodies, semantic dementia and Alzheimer's disease; tauopathy disease; amyotrophic lateral sclerosis (ALS); Parkinson's disease; Spinal muscular atrophy; Pick's disease; Corticobasal syndrome; and normal pressure hydrocephalus.

10. The method of claim 1, wherein the neurological disorder characterized by neurodegeneration is Alzheimer's disease.

11. The method of claim 1, wherein the subject is at risk of developing neurological disorder characterized by neurodegeneration, such as Alzheimer's disease.

12. A kit comprising:
(i) a Rho-associated protein kinase (ROCK) inhibitor or a pharmaceutically acceptable salt thereof; and
(ii) a farnesyltransferase inhibitor or a pharmaceutically acceptable salt thereof.

13. The kit of claim 12, wherein the ROCK inhibitor or a pharmaceutically acceptable salt thereof is fasudil or a pharmaceutically acceptable salt thereof and/or the farnesyltransferase inhibitor or a pharmaceutically acceptable salt thereof is lonafarnib or a pharmaceutically acceptable salt thereof.

14. A pharmaceutical composition comprising:
(i) a Rho-associated protein kinase (ROCK) inhibitor or a pharmaceutically acceptable salt thereof;
(ii) a farnesyltransferase inhibitor or a pharmaceutically acceptable salt thereof; and
(iii) a pharmacologically acceptable excipient and/or diluent.

15. The pharmaceutical composition of claim 14, wherein the ROCK inhibitor or a pharmaceutically acceptable salt thereof is fasudil or a pharmaceutically acceptable salt thereof and/or the farnesyltransferase inhibitor or a pharmaceutically acceptable salt thereof is lonafarnib or a pharmaceutically acceptable salt thereof.

16. The pharmaceutical composition of claim 14, wherein the composition is formulated for intracerebral, intrathecal or nasal administration.

17. A system for assessing the effect of one or more candidate drugs in a neurological disorder animal model comprising:
(a) a transgenic animal model having physiological characteristics associated with a neurological disorder characterized by neurodegeneration;
(b) means for administering said one or more candidate drugs intracerebroventricularly to the transgenic animal model; and optionally
(c) means for obtaining a sample from the transgenic animal model for analysis of one or more biomarkers associated with a neurological disorder characterized by neurodegeneration;
wherein the transgenic animal model has been additionally modified to express exogenous tau protein in neurons.

18. The system of claim 17 which comprises means for obtaining a sample for analysis of one or more biomarkers associated with a neurological disorder characterized by neurodegeneration, wherein the system is configured to enable administration of said one or more candidate drugs and obtaining a sample for analysis simultaneously.

19. The system of claim 17, wherein the transgenic animal model is a transgenic mouse.

20. The system of claim 17, wherein the transgenic animal model:
(a) overexpresses an APP gene comprising the Swedish APP gene mutation (APP_{SWE});
(b) overexpresses a microtubule associated protein tau (MAPT) comprising the P301 L gene mutation (MAPTP₃₀₁L); and
(c) comprises the mutant M146V in the Presenilin-1 (PSEN1) gene (PSEN1_{M146V}).

21. A method for assessing the effect of one or more candidate drugs in a neurological disorder animal model comprising:
(a) providing a transgenic animal model having physiological characteristics associated with a neurological disorder characterized by neurodegeneration;
(b) modifying the transgenic animal model of (a) to express exogenous tau protein in neurons;
(c) administering said one or more candidate drugs intracerebroventricularly to the transgenic animal model; and
(d) analysing one or more biomarkers associated with a neurodegenerative disease in a sample obtained from the transgenic animal and/or assessing the cognitive ability and/or behaviour of the transgenic animal, thereby assessing the effect of the one or more candidate drugs.

22. The method of claim 21, wherein the sample obtained from the transgenic animal for analysing one or more biomarkers is obtained contemporaneously with step (c).

23. The method of claim 21, wherein the transgenic animal model is a transgenic mouse.

24. The method of claim 23, wherein the transgenic animal model:
(a) overexpresses an APP gene comprising the Swedish APP gene mutation (APP_{SWE});
(b) overexpresses a microtubule associated protein tau (MAPT) comprising the P301 L gene mutation (MAPTP₃₀₁L); and
(c) comprises the mutant M146V in the Presenilin-1 (PSEN1) gene (PSEN1_{M146V}).
